(19) United States
(12) Patent Application Publication
(10) Pub. No.: US 2024/0085653 A1
(43) Pub. Date: Mar. 14, 2024

(54) FIBER-OPTIC CONTROLLER AND METHOD

(71) Applicant: Cyclone Biosciences, LLC, Phoenix, AZ (US)

(72) Inventor: Stephen E. Griffin, Peoria, AZ (US)

(73) Assignee: Cyclone Biosciences, LLC, Phoenix, AZ (US)

(21) Appl. No.: 18/228,983

(22) Filed: Aug. 1, 2023

Related U.S. Application Data
(60) Provisional application No. 63/405,984, filed on Sep. 13, 2022.

(51) Int. Cl. G02B 6/44

(52) U.S. Cl. CPC G02B 6/4439; G02B 6/02

(57) ABSTRACT

A holder of an optical fiber (a fiber-optic element) structured to house and secure the surgical optical fiber within the holder to prevent a possibility of the fiber to move at least axially and/or rotate about the axis while, at the same time, avoiding microbending of the surgical optical fiber (which such holder is configured to house) to prevent fiber burning during use of the surgical fiber with high-power laser sources. The holder is optionally and preferably configured as a controller of spatial orientation of the fiber output portion (which output portion remains invisible to the user during the application) that ensures that the output optical power is delivered from the termination of the optical fiber in the intended direction.

### CROSS-REFERENCE TO RELATED APPLICATIONS

The present US patent application claims priority from and benefit of the U.S. Provisional Patent Application No. 63/405,984 filed on Sep. 13, 2022, the disclosure of which is incorporated by reference herein.

### TECHNICAL FIELD

The present invention relates to holding arrangements for securing a surgical optical fiber and, more particularly, to a fiber optical holder structured to avoid microbending of the surgical fiber caused by such securing thereby removing the cause of fiber burning during its use at high optical power.

### RELATED ART

Surgical lasers—such as, for example, holmium lasers or thulium fiber lasers—primarily find applications in urology and, specifically, in operation of vaporization and enucleation of hyperplastic prostate tissue (BPH) and breaking apart kidney stones (although additional applications are known for both soft and hard tissue targets). These infrared lasers typically produce 0.2 Joule to 6 Joule pulses with 250 ρs to 1200 ρs pulse widths at rates from about 5 pulses per second (pps) to about 120 pps at wavelengths ranging from about 1.93 μm to about 2.14 μm, with average powers ranging from about 8 W to about 140 W. The generated laser radiation output is conventionally delivered to the target tissue with the use of an optical fiber-based device judiciously designed to withstand the channeled laser power and to deliver it to the target tissue without complications. In some cases, such optical fiber-based device includes so-called optical fiber terminations structured to outcouple light from the optical fiber in a direction transverse to the fiber axis. The operation of these devices requires, among other degrees of freedom, the reliable holding/positioning and rotation of the optical fiber while, at the same time, the ability to know or estimate how such the holding and rotation affects the direction and orientation of the laser light output towards the tissue in real time.

### SUMMARY OF THE INVENTION

Embodiments of the invention provide an article of manufacture that includes a fiber-optic controller. The fiber-optic controller includes a handle and a nose cap. The handle has a handle axis and a handle hollow (that stretches and is defined throughout the handle along the handle axis). The handle includes a collet with petals that extend along the handle axis and that have respectively-corresponding free distal ends and proximal ends. (Here, inner surfaces of the petals are separated from the first axis at locations of the free distal ends by a first distance and inner surfaces of the petals are separated from the first axis at locations of the proximal ends by a second distance smaller than the first distance.) The nose cap has a nose cap axis and a nose cap hollow defined throughout the nose cap and extending along the nose cap axis. At least a portion of the nose cap hollow is configured as a cavity extending along the nose cap axis and dimensioned such that—when the handle and the nose cap are mated by inserting the free distal ends of the petals into the cavity and positioning a ridge of a first surface into a corresponding notch on a second surface—the free distal ends are brought towards one another to change the first distance to become substantially equal to the second distance. (Here, the first surface is one of an inner surface of the cavity and an outer surface of the handle while the second surface is the other of the inner surface of the cavity and the other surface of the handle.) In at least one implementation, the ridge of the first surface is an arcual ridge as seen in a first plane transverse to a corresponding axis of the first and second axes, and the notch is a groove as seen in a second plane transverse to the other of the first and second axes. In one specific case, each of the ridge and the notch may be structured as parts of corresponding threads (located at the nose cap and handle, respectively). Alternatively, in a specific implementation of the fiber-optic controller that is devoid of a thread, each of the ridge and the notch may be spatially extended along the circumference of the corresponding surfaces of the nose cap and the handle at a pre-defined angle (as seen along an axis of the corresponding portion of the fiber-optic controller) that ranges from a value exceeding zero degrees to a value of substantially equal to 360 degrees. When the ridge/notch are not part of a corresponding thread, the action of position the ridge into the notch is performed by snapping one into another. At least in one implementation, the cavity may be dimensioned such that, when the handle and the nose cap are mated, a distance between an inner surface of a petal of the collet and the first axis is substantially constant at every point of said inner surface along the first axis for a given angular orientation in a plane perpendicular to the first axis, and/or each of the petals includes longitudinal ridges on a corresponding inner surface thereof (with such longitudinal ridges having lengths that are substantially equal to a length of such petal; here, the longitudinal ridges are dimensioned such that—when a given optical fiber is pulled throughout the handle hollow and throughout the nose cap hollow and when the handle and the nose cap are mated—the longitudinal ridges are in contact with a plastic coating of the optical fiber substantially uniformly along the lengths of the longitudinal ridges while, at the same time, not being in contact with a cladding of the optical fiber. Substantially in every implementation, the fiber-optic controller may be structured to be devoid of a thread, if so desired, and/or is configured i) to have a handle surface indicia on an outer surface of the handle, such handle surface indicia configured to cause the outer surface of the handle to be asymmetric with respect to rotation about the handle axis; and/or ii) to have a nose cap surface indicia on an outer surface of the nose cap, such nose cap surface indicia configured to cause the outer surface of the nose cap to be asymmetric with respect to rotation about the nose cap axis. Substantially in every implementation, the article of manufacture may additionally include an optical fiber termination device permanently cooperated with a distal end of the optical fiber such that the optical fiber termination device is separated from the fiber-optic controller by a non-zero length of the optical fiber. In one specific case, the optical fiber termination device may be configured to define an output axis along which light from the distal end of the optical fiber is outcoupled substantially transversely to the optical fiber.

Embodiments of the invention further provide a method for using an implementation of an article of manufacture described above. In particular, an embodiment of the method includes a step of cooperating a handle (which has a collet having a length and containing an optical fiber therein) with a nose cap (that includes a throughout longitudinal hollow that contains the optical fiber therein) such as to position petals of the collet inside the hollow of the nose cap. The method further includes a step of securing the nose cap onto the handle to form an embodiment of the fiber-optic controller that has been described above and that contains the optical fiber by mutually repositioning the handle and the nose cap towards one another to snap a ridge, located on a first surface, into a corresponding notch located on a second surface. As a result of such securing, a plastic coating of the optical fiber is brought in contact with an internal surface of the collet substantially uniformly along the length while, at the same time, a cladding of the optical fiber is not brought in contact with the collet. (Substantially in every implementation of the method, the bringing of the internal surface of the collet in contact with the plastic coating of the fiber may include squeezing the plastic coating with longitudinal ridges of the inner surface of the collet without a longitudinal ridge of the longitudinal ridges touching the cladding to displace the plastic coating to provide resistance to rotation of the optical fiber inside the collet and/or to rotation of the collet about the optical fiber substantially at every point of the length.) Here, the first surface is one of an inner surface of the hollow of the nose cap and an outer surface of the handle while the second surface is the other of the inner surface of the hollow of the nose cap and the other surface of the handle. Substantially in every implementation of the method, at least one of the following conditions may be satisfied: (i) the step of cooperating includes inserting the petals of the collet substantially completely into a first portion of the hollow of the nose cap while leaving a second portion of the hollow of the nose cap to contain only the optical fiber; (ii) the step of the securing does not require a rotational movement of the nose cap and/or of the handle about the optical fiber; and (iii) the step of securing is necessarily devoid of said rotational movement.

### BRIEF DESCRIPTION OF THE DRAWINGS

The invention will be more fully understood by referring to the following Detailed Description of Specific Embodiments in conjunction with the Drawings, of which:

FIGS. 1A, 1B, 1C, 1D illustrate a typical fiber optic holder of related art.

FIG. 2 is an exploded isometric view of an embodiment of the invention.

FIG. 3 is an exploded cross-sectional view of the embodiment of the invention of FIG. 2.

FIG. 4 is a cross-sectional view of the embodiment in its assembled form.

FIGS. 5A, 5B and 6A, 6B, 6C, 6D illustrate the assembled embodiment at different viewing angles.

FIGS. 7A, 7B, 7C, and 7D provide several views of a portion of a single petal of the collet of the embodiment, equipped with judiciously dimensioned ridges on the inner surface to grasp and displace a polymeric coating of the optical fiber without touching the surface of the glass coating of the fiber.

FIGS. 7E, 7F illustrate the affixation of the optical fiber element within the collet of the handle of an embodiment of the invention.

FIGS. 8A, 8B are axial views of a handle of the embodiment with the petals of the collet in a closed position, in which the axial opening surrounded by the petals has cross-sectional dimensions that are maintained substantially unchanged along all of the lengths of the petals.

FIGS. 9A, 9B provide views of an embodiment of the nose cap portion of the fiber-optic controller.

FIGS. 10A, 10B show a dissected along its axis assembled embodiment of the invention with the optical fiber passing throughout the handle hollow and throughout the nose cap hollow (while the handle and the nose cap are mated) and secured in its position and angular orientation at least by the collet of the handle portion of the embodiment. FIG. 10A: a view showing both “halves” of the dissected embodiment; FIG. 10B: a close-up view, illustrating a portion of the optical fiber squeezed by the petals of the collet of the handle of the embodiment and demonstrating that, when the optical fiber is pulled throughout the handle hollow and throughout the nose cap hollow and when the handle and the nose cap are mated, the longitudinal ridges (on the inner surface of the petals) are in contact with a plastic coating of the optical fiber substantially uniformly along the lengths of the longitudinal ridges while, at the same time, not being in contact with a glass cladding of the optical fiber.

FIG. 11 illustrates an embodiment of the surgical optical fiber-based device equipped with an embodiment of the fiber controller configured according to the idea of the invention.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another. Drawings are generally not to scale.

### DETAILED DESCRIPTION

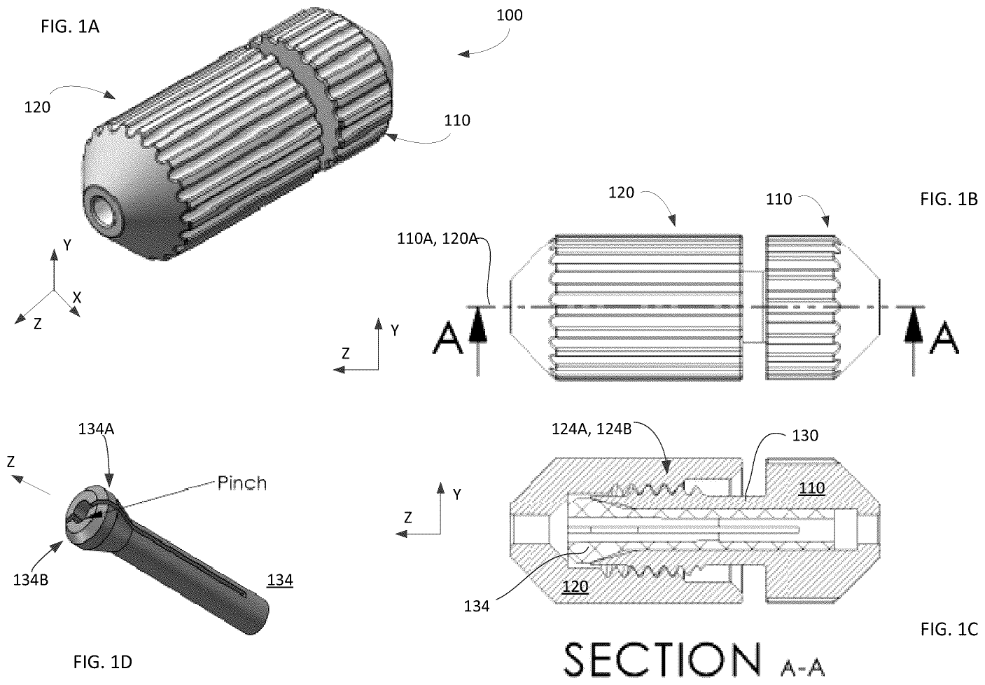

Multiple holders or handles for an optical fiber are utilized in related art, the operation of a substantial number of which turns on securing the optical fiber in a pin vise like contraption having a collet structured such that the petals of the collet close upon and secure the fiber, drawn through the collet, in response to a nut or nose cap being screwed onto a thread located radially farther from the axis of the contraption than the collet (an, in some cases, on the outer side of the collet itself). Pin vises, as the name suggest, hold thin, long cylindrical objects (like a pin or wire) by one end. Similar structures have been widely utilized to successfully secure a drill bit in a drill bit adapter—such as a pin chuck vise, for example. In other words, skilled persons understandably and logically think of securing the optical fiber in a holder structured by analogy with a pin vise since securing a drill bit in one—and reliably utilizing the so-secured drill bit—has been the industrial staple for the longest time. One of various examples of the holder/affixer of an optical fiber of related art, structured according to the principle of a pin vise, is schematically illustrated in FIGS. 1A, 1B, 1C, and 1D. As shown, the conventional holder 100 may include a handle 110 and a nose cap 120 (each having a corresponding throughout hollow formed along the corresponding axis 110A, 120A), which are mated with one another via corresponding threads 124A, 124B. In this case, as shown, one thread is formed on an inner surface of the nose cap 120, while the reciprocal other—on an outer surface of the cylindrically-shaped protrusion 130 at the front end of the handle 110. The collet 134 (shown with two petals 134A, 134B, dimensioned to have a radially-outwardly expanding outer surface at one end, thereby forming a bulge at this end) is configured to slidingly fit inside the hollow of the protrusion 130 such that, when the collet 134 is installed inside this hollow 130 with the bulge positioned on the outside and when the nose cap 120 is screwed onto the thread of the handle—as shown in FIGS. 1B, 1C, the petals of the collet are squeezed towards one another (that is, repositioned towards the axes 110A, 120A). Understandably, if the optical fiber has been pulled through the hollows of the handle, collet, and the nose cap prior to threading of the nose cap onto the protrusion of the handle, the optical fiber is at least partially fixated inside the collet as a result of the petals grasping it at the bulging end.

As far as using this or a similar implementation of the optical fiber holder in laser surgery is concerned, however, a rather substantial problem arises: together with being affixed inside the holder, the polyimide or other plastic-type jacket (for example, an engineering plastic with high performance, ETFE) of the fiber and the glass body of the fiber are being pinched at least at the location corresponding to ends of the petals and, therefore, the fiber becomes/is microbent. A person of skill in the art immediately recognizes now how the intractable microbending can detrimentally affect the operation of the surgical fiber in practice: at 100 watts of laser output, carried by the fiber in multiple spatial modes, the fiber simply burns through at the pin vise pinch point if the pin vise is tightened enough to control the fiber without hysteresis.

Practice shows that, generally, substantially all conventionally structured optical fiber holders employed for use with a surgical optical fiber pinched the fiber too much, causing it to burn through, with a resounding pop. (Consider, for example, a conventional used of a surgical 2.080 um laser at 4 J per pulse, 250 us pulse widths, and a typically low beam quality at M² of 400 or higher. The M² factor of a laser beam is often referred to as a beam quality factor or beam propagation factor, and is a common measure of the beam quality of a laser beam and is known in related art to represent the degree to which the light beam can be focused for a given beam divergence angle. A diffraction-limited beam such as a Gaussian beam, for example, has an M² factor of 1. A typical value of M² for a surgical laser such as a holmium laser is at least several tens, sometimes less than 50, but when such laser is overheated—which is a common occurrence—the M 2 value can reach triple digits: thermally-induced refractive index gradients and birefringence in holmium laser rods distort the laser output, both beam diameter and divergence drift during use/operation of a given laser, and myriad spatial modes are generated.)

Notably, the use of a modified collet (for example, a collet of an E type that possesses cuts/slit between the petals from both ends to near the opposite ends so that both ends are compressed substantially equally) in an optical fiber holder that employs a rotation of one holder component with respect to another to bring such collet to its closed state was shown to still result in fiber microbending, as such type of collet effectively produces two fiber pinch locations (one at each end of the collet) but some additional, lesser force contact between the ends. As a result, the optical fiber is still not equally radially compressed or squeezed at each point within the collet. As the skilled person appreciates, the market for the surgical fibers has recently substantially morphed to include the fibers capable of handling optical outputs from even higher-power holmium lasers that offer up to 6 Joules at 150 Watts of average power, which exacerbates the problem further. This begs the question of devising a fiber holder that, upon affixation of the fiber passing through it, does not microbend this fiber.

A problem of a conventional fiber-optic holder (the one configured to secure an optical fiber therein as a result of rotational movement of one controller part with respect to another controller part) manifesting at least in pinching the optical fiber at at least one point along the length of the controller and thereby causing a microbending of the optical fiber—is solved by devising a fiber-optic holder that is preferably devoid of a thread and/or does not require a rotational movement to secure the optical fiber. A corollary deficiency of the conventional fiber-optic holder that is its inability to identify and/or indicate a preferred radial direction of a fiber-optic article secured in such holder and, therefore, to operate as a fiber-optic controller, is solved by dimensioning the proposed fiber-optic holder to be substantially rotationally asymmetric and, when needed, securing an optical fiber in such holder with the preferred radial direction substantially aligned with a feature causing asymmetry of the fiber-optic holder.

From the following description the skilled person will recognize that one of the practical structural characteristics of proposed embodiments is that the petals of the collet of the embodiment form such a hollow axial passage (surrounded by the petals) the maximum cross-sectional dimension of which is necessarily slightly larger than the fiber plastic jacket's (ETFE's outer diameter) outer diameter but the minimum cross-sectional dimension of which is necessarily slightly smaller than the EFTE's outer diameter while, at the same time, remaining larger than the diameter of the outer glass later of the fiber—and maintains these dimensions substantially the same over the length of the collet. This feature is achieved, in at least one implementation, by structuring the inner surface of the petals to possess ridges or rims extending axially along the whole length of the petals and, preferably, distributed substantially uniformly when seen along the axis. As a result, upon the closure of the petals onto and over the optical fiber pulled through the hollow of the collet, the ridges “bite” into the plastic/polymer jacket of the fiber without reaching the glass surface and displace the polymeric material of the jacket in spaces between the ridges for the substantially net zero compression of the glass body of the fiber yet providing a firm grip on the fiber to prevent its rotation within the collet (which is a rather critical advantageous characteristic required in use of the surgical fibers) and/or axial repositioning of the fiber as a result of longitudinal push and/or pull movements. Further, it is understood that as a result of avoiding a rotational movement of one part of the holder with respect to another during the closing of the collet onto the fiber the resulting closure is, substantially, fixed: there is no overtightening possible because there is but one open position of the collet and one closed position of the collet.

A skilled person will also appreciate that, in practice, a measure of the microbending stresses on the fiber core (or lack thereof) can be devise simply by looking at the divergence of the light output from the fiber when provided with a minimally divergent light output (such as that from the HeNe laser): the divergence substantially approaching zero is a clear indication of the lack of microbending stresses in the fiber.

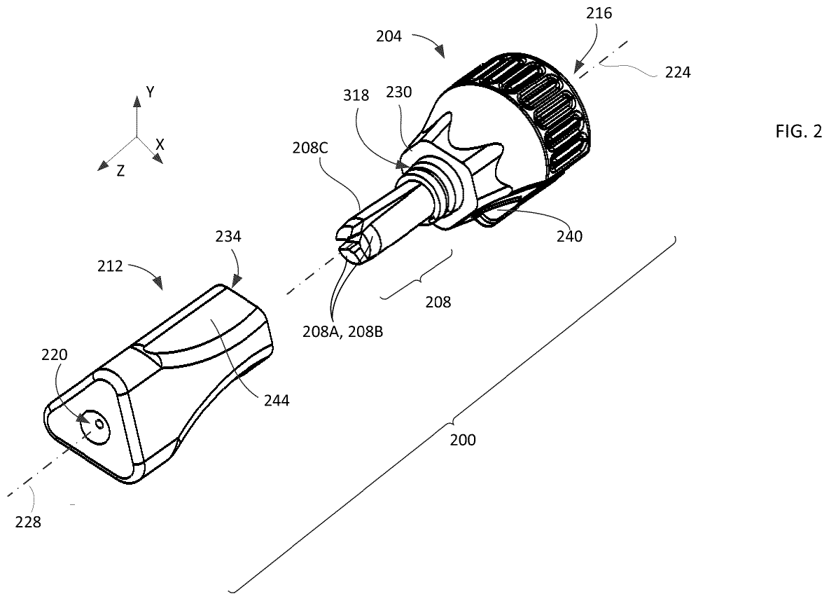

The description of a specific but non-limiting in structure and function embodiment of the invention is now provided in reference to FIGS. 2, 3, 4, 5, 6, 7A-7E, 8A-8B, 9A-9B, 10, and 11.

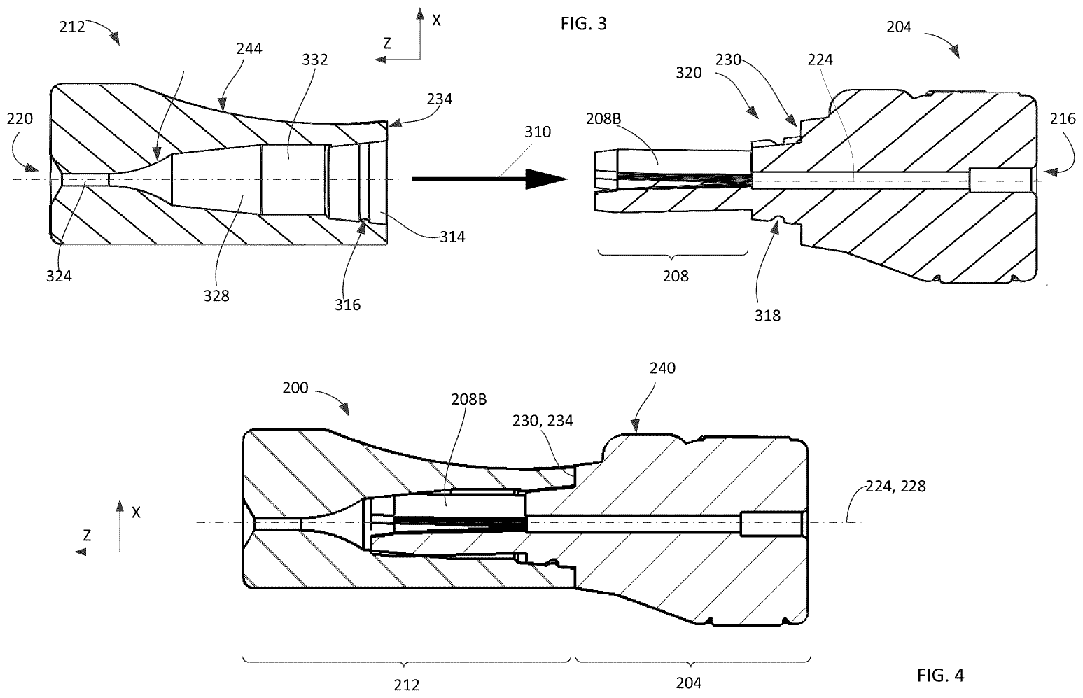

FIG. 2 provides an isometric exploded view of an embodiment 200 of the invention, showing a handle 204 having a collet 208 (in this example, the collet 208 is shown to have three petals 208A, 208B, 208C) and a nose cap 212. FIG. 3 illustrates the same in cross-sectional views, while FIG. 4 presents the cross-sectional view of the assembled embodiment.

Both the handle 204 and the nose cap 212 have corresponding internal throughout hollows 216, 220, formed along corresponding axes 224, 228 of the handle and the nose cap (and clearly seen in the cross-sectional views of FIG. 3). In a disassembled stated of the embodiment 200 (shown in FIG. 2), the collet 208 is in an open state when the free distal ends of the petals 208A, 208B, 208C (facing the nose cap 212 in FIG. 2) are radially separated from the axis 224 by a distance that is larger than a distance separating the opposite proximal ends of the petals (that is, the petal ends at which the collet 208 is rooted in the neck (shown as 320 in FIG. 3) of the handle 204) from the axis 224. The state of the collet 208 is changed from open to closed by roughly aligning the axes 224, 228 with one another and bringing the handle 204 and the nose cap 212 closer together with a translational motion along the axes (see the indicating arrow 310 in FIG. 3) while inserting the petals 208A, 208B, 208C into the hollow 220 through the wide mouth opening 314 of the hollow 220 until the handle and the nose cap are mated.

The mating is accomplished when a ridge 316 on the internal surface of the hollow 220 snaps into a reciprocally dimensioned notch 318 on the outer surface of the neck 320 of the handle 204 (and in at least one case—as shown in FIG. 3—the surfaces 230 of the handle 204 and 234 of the nose cap 212 are substantially brought in contact when the mating of the pieces 204, 212 is accomplished). In at least one implementation, the inner surface of the hollow 320 (on which the ridge 316 is formed) and the outer surface of the neck 320 may be generally dimensioned as conical surfaces. The ridge 316 may be shaped to be arcual about the axis 228 and, in at least one case, the ridge 316 may be formed to be circumferential about the axis 228. (In the latter case, understandably, it is advantageous to have the notch 318 be structured as a groove that is circumferential about the axis 224.)

According to the idea of the invention, the embodiment 200 is devoid of (does not contain) a thread and, while mating the components 204, 212, no rotational movement about the axes 224, 228 is required and/or performed at all: in at least one specific case, the mating between the components 204, 212 is accomplished only with a longitudinal, axial movement of the components in absence of rotation of any of the components 204, 212 about its corresponding axis. The hollow 220 of the nose cap 212 is generally configured to be multi-sectional (see, for example, sections 324, 328, 332 that differ from one another in dimensions). The section of the hollow 220 accommodating the free ends of the petals of the collet 338 necessarily has a conical surface dimensioned such as to gradually, in a monotonic fashion bring the free ends closer together as the collet is being pushed into the hollow 224 (through the mouth opening 314) up to the point that—when the cooperation of the ridge 316 and the notch 318 occurs and the insertion movement stops and the components 204, 212 are mated—the radial distances between the axis 224 and the chosen points on the inner surfaces of the petals at the proximal ends thereof are the same as the radial distances between the axis 224 and the corresponding chosen points on the inner surface of the petals at the free distal ends thereof. In other words, the inner hollow 220 of the nose cap 212 is judiciously dimensioned to monotonically deflect the free distal ends of the petals from their open state towards one another and to ensure that, upon completion of the mating, the inner surfaces of the petals 208A, 208B, 208C are substantially tangentially-parallel to one another.

Since the functional purposes of the embodiment 200, according to the idea of the invention, is not only to hold the optical fiber pulled through the hollows 216, 220 and affixed therein upon the completion of the mating between the handle 204 and the nose cap 212 but also for the user to be certain about the angular orientation of a reference portion of the fiber when the embodiment 200 is rotated about its axis with the fiber affixed therein (that is, to perform a function of optical fiber controller), at least one of the handle 204 and the nose cap 212 is made substantially rotationally asymmetric and/or contains a predefined indicia configured as a reference with respect to which the reference portion of the optical fiber is positioned prior to the assembly of the embodiment. For example—as shown in FIGS. 2, 3, 4—the handle 204 is dimensioned to be asymmetric with respect to a rotation about its axis 224 and contains a cuticle 240 extended along the outer surface of the handle 204.

This asymmetric structural feature may be employed, for example, when the embodiment 200 is used with a surgical optical fiber equipped, at the distal end, with a termination structured to re-direct light propagating inside the fiber sideways (in which case the resulting optical-fiber-based device is known to a skilled person as a side-fire fiber device, see for example U.S. Pat. Nos. 9,323,005, 7,909,817, 6,687,436, 5,562,657, the disclosure of each of which is incorporated herein by reference). Then, prior to mating of the handle and nose cap portions of the embodiment of the invention to secure the optical fiber (with the termination) that has been pulled throughout the hollows 216, 220, the handle may be oriented to spatially coordinate the angular position of the cuticle 240 about the axis to that of the direction along which the output light is delivered from the termination. Alternatively, or in addition, the structural asymmetry of the embodiment may be employed when the optical fiber is a polarization maintaining fiber. Here, the orientation of one of the axes (fast or slow) or such fiber may be spatially coordinated with the cuticle 240 prior to the assembly of the embodiment 200.

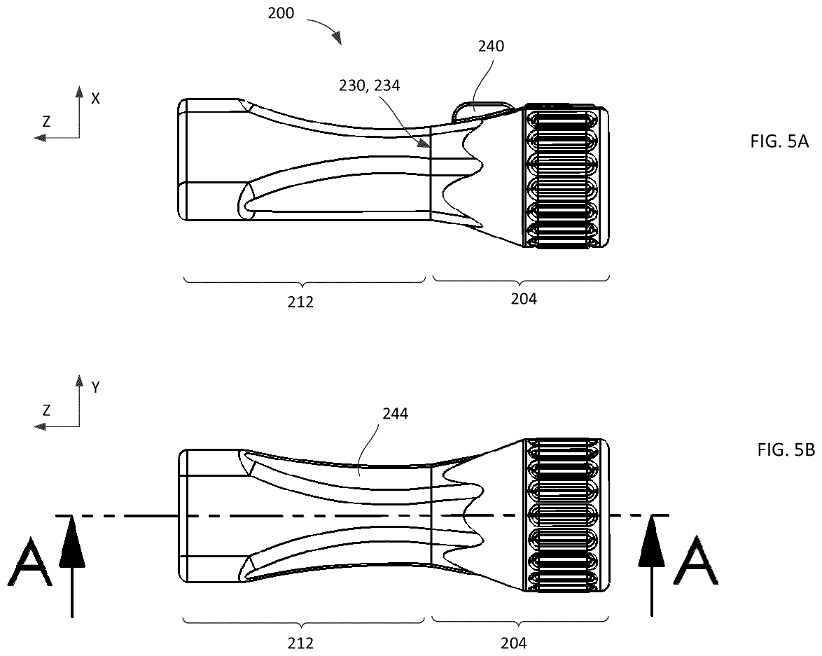

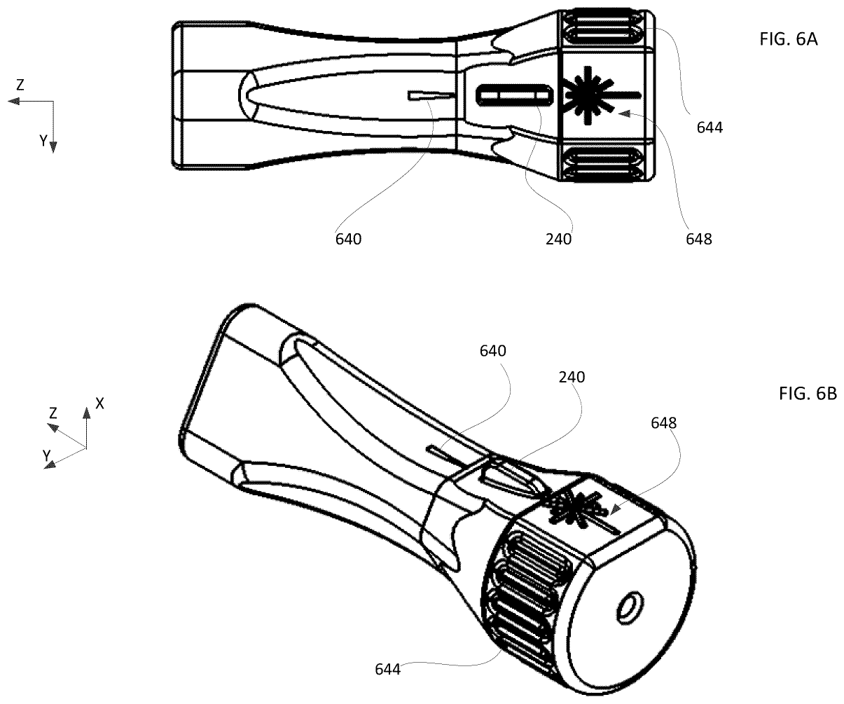

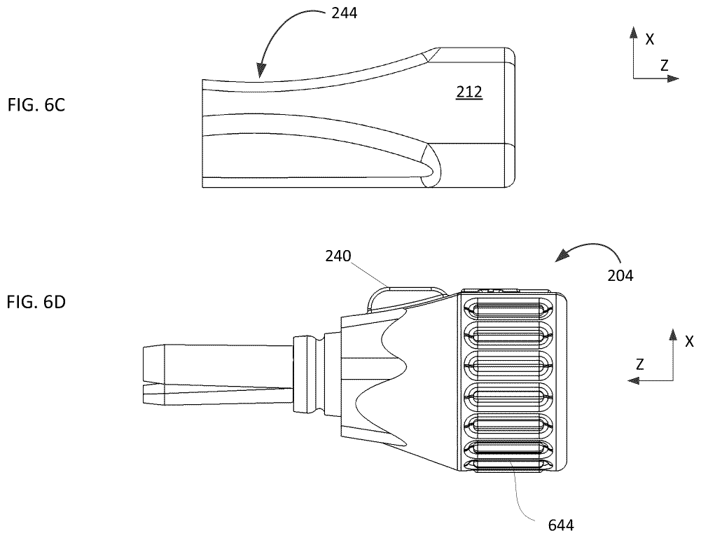

In one specific implementation, the outer surface of the nose cap 212 may include at least one longitudinal facet 244. The presence and number of the facets is preferably decided upon depending on the use of the embodiment of the 200. For example, when an embodiment of the invention is configured to be used in a very specific application—a laser resection of the prostate (a gland with three lobes about the urethra)—three facets 244 (just as shown in FIGS. 5A, 5B, 6A, 6B) are formed, which is intended to provide a level stability in the center of each lobe of the target gland.

FIGS. 5A and 5B illustrate an embodiment 200 of the assembled contraption in front and bottom views, while FIGS. 6A, 6B provide a top view and a perspective view, respectively. As shown in FIGS. 6A, 6B, embodiment may optionally contain additional indicia 640 (such as a ridge or a groove, in one specific case, that is axially aligned, when assembled, with the cuticle 240) and/or ornamental surface relief 644 (already indicated in FIGS. 5A, 5B) and/or additional indicia 648 on a facet formed on an outer surface of the handle 204. Notably, the view of FIG. 3 represents the cross-section in plane A-A of FIG. 5B. FIGS. 6C, 6D provide additional side views of the portions 204, 212 of the embodiment of the invention.

Referring again to FIGS. 2, 3, and 4, preferably, for certainty of the assembly of the embodiment, the handle 204 and the nose cap 212 are configured such that, when mated, the facing each other base surfaces of these components are substantially brought in contact with one another. The minimal diameters of the hollows 216, 220 are dimensioned to accommodate a target surgical optical fiber element therein, together with the outer polymeric jacket of such fiber element but such as to not have the glass body of the fiber pinched at any point of the internal surfaces of the petals.

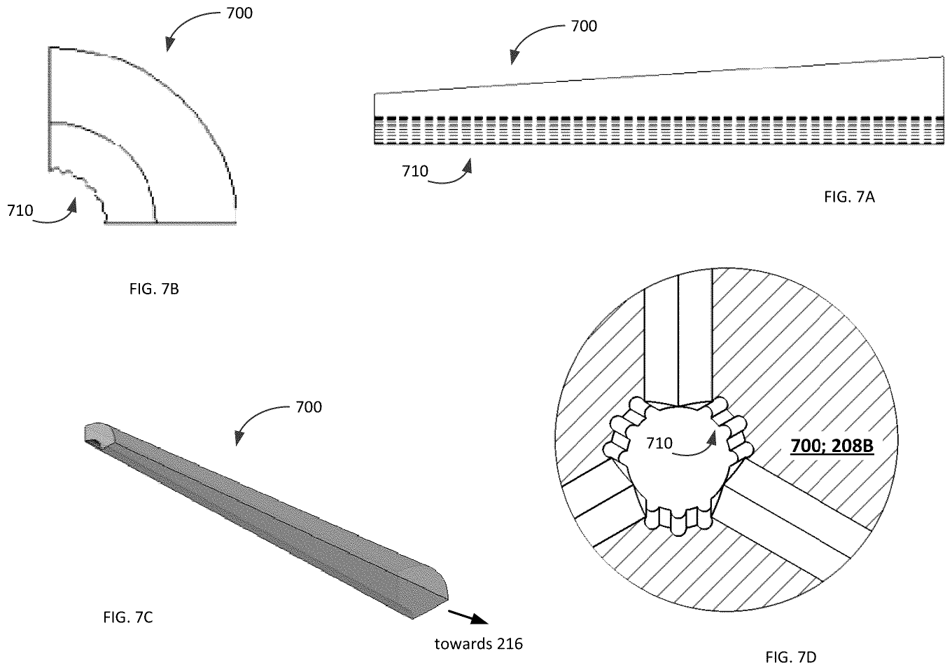

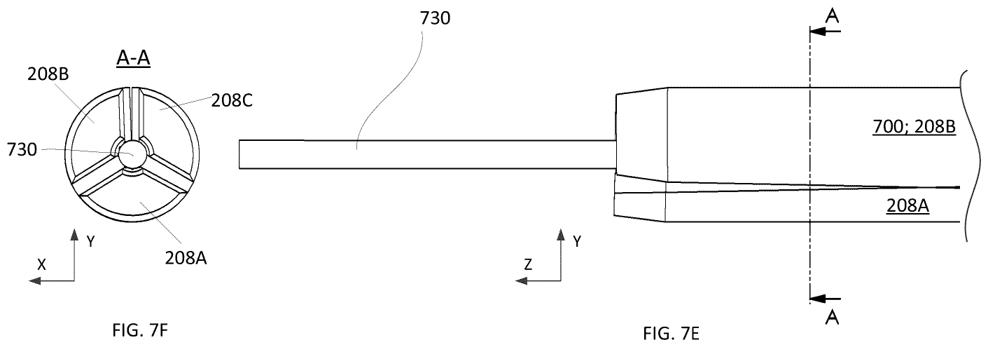

To this end, according to the idea of the invention and in reference to the schematics of FIGS. 7A, 7B, 7C, and 7D, each of the petals present in a given implementation of the collet (one of which, separated or “broken off” from the body of the handle 204, is illustrated as 700) is configured to have a plurality of ridges 710, formed on the inner surface thereof and extending along the length of a particular petal FIG. 7A shows schematically a side view of a single petal, FIG. 7B provides a front view of it, FIG. 7C presents a schematic isometric view, and FIG. 7D shows an axial portion of the collet 208 (viewed against the z-axis) in closed position. FIG. 7D clearly identifies the presence of ridges of “teeth” 710 of two different heights, as discussed below.

FIG. 7E presents schematically a collet portion of the handle 204, illustrating the optical fiber element 730 pulled through the hollow 216 of the handle 204, while FIG. 7F shows a cross-sectional view of the same with plane A-A.

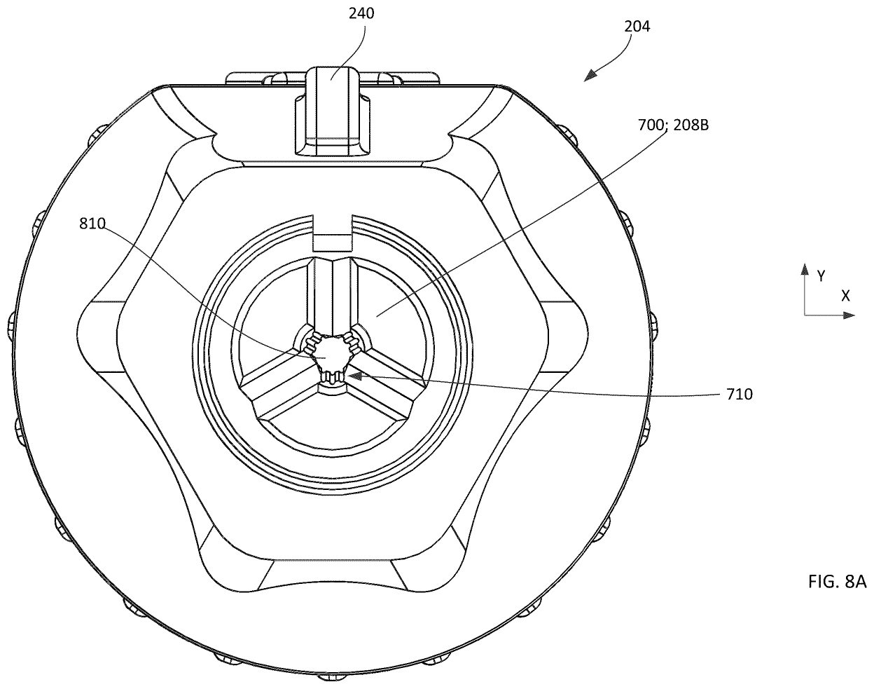

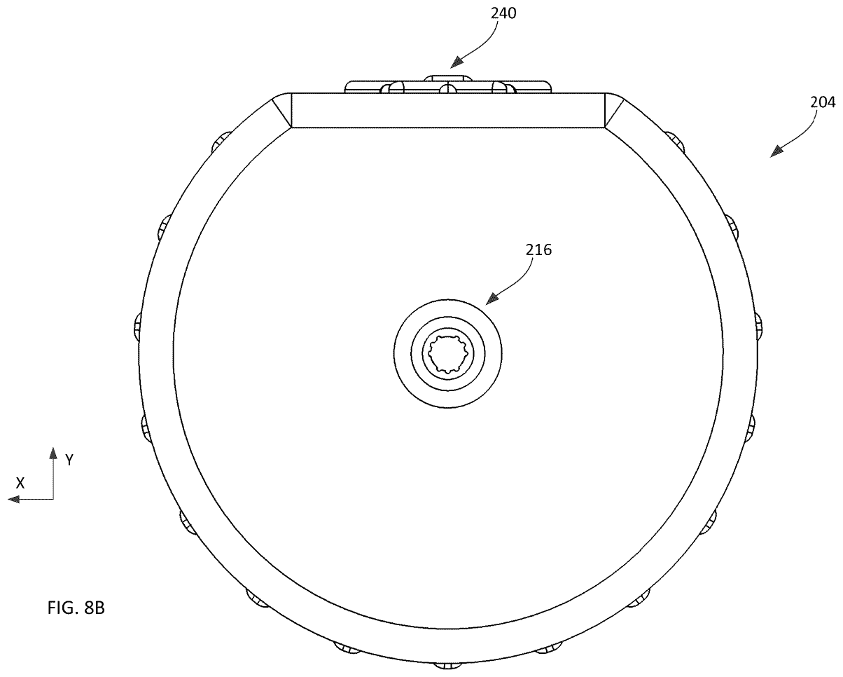

FIG. 8A shows the handle 204 as viewed against the z-axis (that is, in the −z direction) with the collet 208 being in the closed position or state. The numeral 810 indicates the axial opening formed by the inner surfaces of the petals 208A, 208B, 208C in the closed state of the collet 208 (which is a part of the hollow 216). As seen, a portion of the hollow axial passage 216 has the maximum cross-sectional dimension (defined by sections of the opening between the ridges 710) that is dimensioned to be necessarily slightly larger than the fiber plastic jacket's (ETFE's outer diameter) outer diameter of the target surgical fiber to be pulled through the hollow 216. At the same time, the minimum cross-sectional dimension of the opening 810 (defined by the ridges 710) is necessarily slightly smaller than the EFTE's outer diameter (and, at the same time, larger than the diameter of the outer glass layer of the fiber.

The ridges 710 are judiciously dimensioned to partially displace the polymeric material of the ETFE polymer jacket of the optical fiber. In one example, the ETFE is 0.18 mm thick and has a 1.100 mm outer diameter. The maximum diameter of the opening 810 is about 1.150 mm and—with the height of the ridges being about 0.1 mm—the ridges displace a about 0.050 mm of the thickness of the portion of the EFTE layer. The displaced polymer flows or transitions into and at least partially fills the spaces between the ridges to provide resistance to axial rotation of the fiber with minimal (if at all) localized radial pressure produced on the glass material of the fiber.

To phrase it differently, upon the closure of the collet 208 onto and over the optical fiber pulled through the hollow of the collet, the ridges 710 “bite” into the plastic/polymer jacket of the fiber without reaching the glass surface and displace the polymeric material of the jacket in spaces between the ridges for the substantially net zero compression of the glass body of the fiber yet providing a firm grip on the fiber to prevent its rotation within the collet (which is a rather critical advantageous characteristic required in use of the surgical fibers) and/or axial repositioning of the fiber as a result of longitudinal push and/or pull movements.

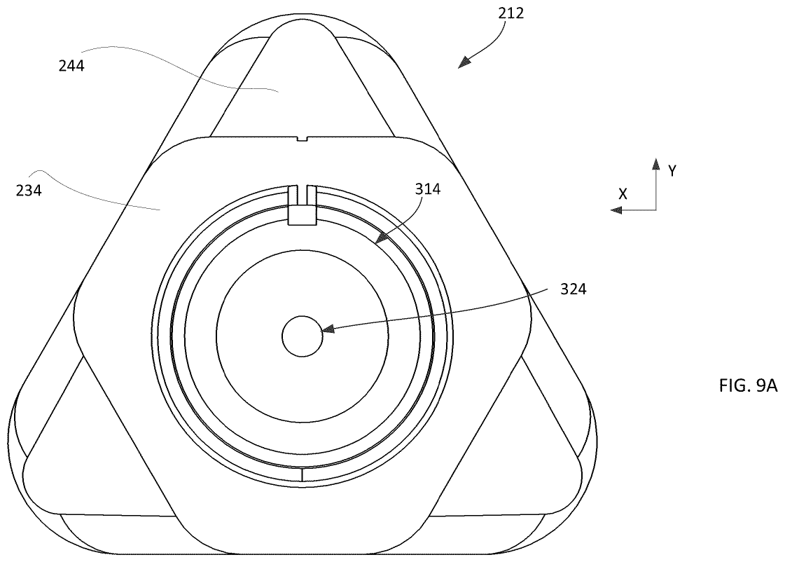

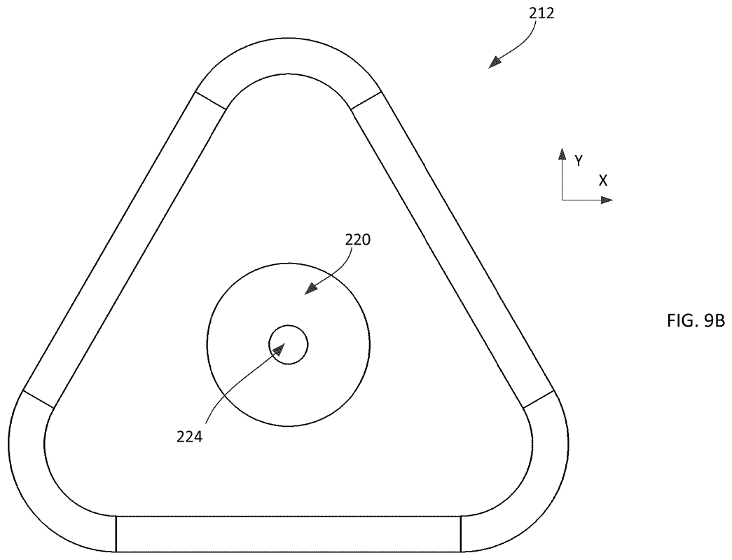

FIG. 8B complements FIG. 8A by illustrating the handle 204 from the end configured for convenient entry of the optical fiber element into the hollow 216—that is, from the end opposite to that illustrated in FIG. 8A. Similarly, FIG. 9A shows an axial view of the nose cap 212 as seen along the +z axis, from the end of the nose cap into which the collet 2ou is being inserted during the assembly of the overall fiber optic controller, while FIG. 9B illustrates the view of the nose cap 212 from the opposite end.

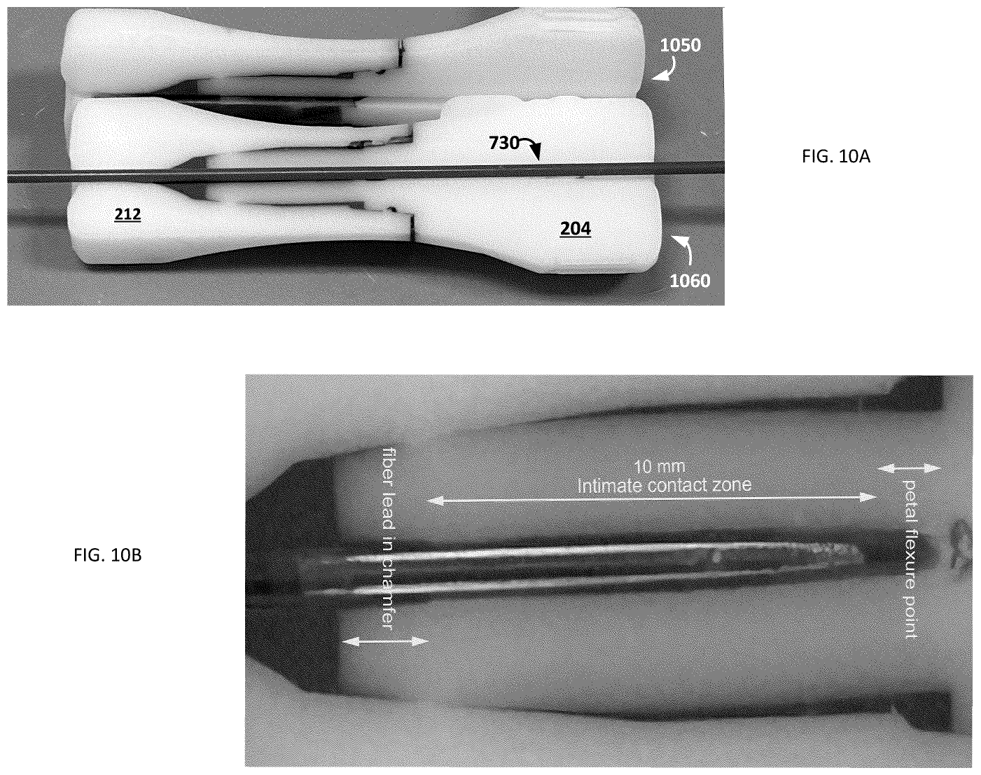

FIG. 10A provides additional illustration by showing, side by side, two halves 1050, 1060 of the assembled version of the embodiment shown in FIG. 4 but containing the optical fiber secured in the embodiment, which assembled version has been dissected, sliced along a plane containing the axes 224, 228 such as to illustrate the affixation of the optical fiber between the petals of the collet of the handle 204 caused by “closing” of the collet due to insertions of the collet into the cavity of the nose cap 212 and securing the closed collet inside the nose cap 212 without microbending the fiber, as discussed above. When the optical fiber is polarization-maintaining, the presence of the surface indicia (such as elements 240, 640) at the handle and nose cap portions of the fiber-optic controller allows the user to properly spatially coordinate the orientation of a fast axis and/or a slow axis of such fiber with respect to the indicia. In one non-limiting case, one of these cases may be intentionally substantially aligned, for example, with at least one of the two indicia thereby providing the user with knowledge of spatial orientation of the polarization vector at the output from such optical fiber. FIG. 10B: a closer-up view similar to that of FIG. 10A, illustrating a portion of the optical fiber squeezed by the petals of the collet of the handle of the embodiment (with the plastic protective coating being partially removed) and demonstrating that, when the optical fiber is pulled throughout the handle hollow and throughout the nose cap hollow and when the handle and the nose cap are mated, the longitudinal ridges (on the inner surface of the petals) are in contact with a plastic coating of the optical fiber substantially uniformly along the lengths of the longitudinal ridges (marked as “the intimate contact zone”; in this case—about 10 mm) while, at the same time, not being in contact with a glass cladding of the optical fiber. Substantially in every implementation, the “intimate contact zone” is t least 4 mm long or longer.

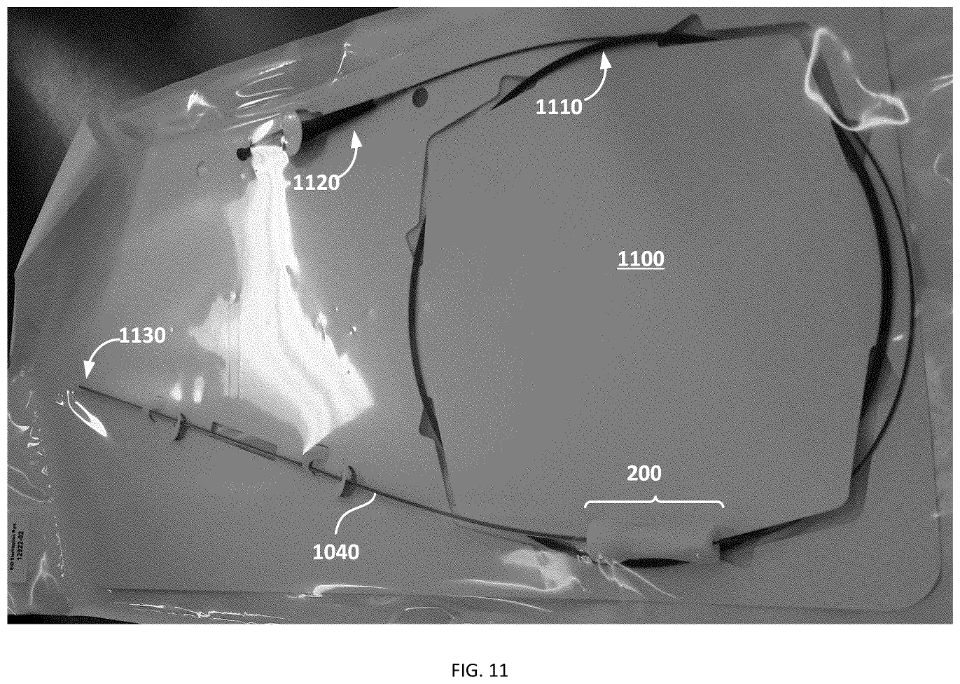

FIG. 11 illustrates an embodiment 1100 of the assembled side-fire optical fiber-based system, in which the embodiment of the fiber controller such as the embodiment 200 can be seen assembled at a location of the (shown coiled) optical fiber 1110 between the location of the fiber connector 1120 (configured for attachment to the laser source) and the optical fiber termination 1130 (which may be optionally structured to direct the light output from the termination in a direction transverse to local axis of the fiber. Optionally, the distal portion of the fiber between the controller 200 and the termination 1130 may be housed in an appropriately structured tube or cannula to provide rigidity to the distal end of the device (as discussed in patent documents incorporated herein by reference, for example). In at least one case, the optical fiber termination may be structured to define an output axis along which light from the distal end of the optical fiber is outcoupled substantially transversely to the optical fiber. In this case, the presence of the surface indicia (such as elements 240, 640) at the handle and nose cap portions of the fiber-optic controller allows the user to properly spatially coordinate the orientation of the output axis of the termination with respect to the indicia. In one non-limiting case, for example, the optical fiber termination may be substantially permanently secured over the distal end of the fiber such that the output axis of the optical fiber termination is substantially aligned with at least one of the two indicia (which is determined when the embodiment 1100 is substantially linearly stretched) thereby providing the user with knowledge of spatial orientation of the direction of the light output at the distal end of the embodiment 1100 as a function of angular rotation of the nose cap and/or the handle about the optical fiber.

The disclosure of each and every document referred to herein is incorporated herein by reference.

For the purposes of this disclosure and the appended claims, the expression of the type “element A and/or element B” has the meaning that covers embodiments having element A alone, element B alone, or elements A and B taken together and, as such, is intended to be equivalent to “at least one of element A and element B”.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention. Within this specification, embodiments have been described in a way that enables a clear and concise specification to be written, but it is intended and will be appreciated that embodiments may be variously combined or separated without parting from the scope of the invention. In particular, it will be appreciated that all features described herein are applicable to all aspects of the invention.

When the present disclosure describes features of embodiments of the invention with reference to corresponding drawings (in which like numbers represent the same or similar elements, wherever possible), the depicted structural elements are generally not to scale, and certain components may be enlarged or reduced in size relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. The use of this term in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated may vary within a range defined by an experimental measurement error that is typical when using a measurement method accepted in the art for such purposes. As an example only, a reference to a vector or line or plane being substantially parallel to a reference line or plane is to be construed as such vector or line extending along a direction or axis that is the same as or very close to that of the reference line or plane (with angular deviations from the reference direction or axis that are considered to be practically typical in the art, for example between zero and fifteen degrees, more preferably between zero and ten degrees, even more preferably between zero and 5 degrees, and most preferably between zero and 2 degrees). For example, the terms “approximately” and about”, when used in reference to a numerical value, represent a range of plus or minus 20% with respect to the specified value, more preferably plus or minus 10%, even more preferably plus or minus 5%, most preferably plus or minus 2%.

The invention as recited in claims appended to this disclosure is intended to be assessed in light of the disclosure as a whole, including features disclosed in related art to which reference is made.

While the invention is described through the above-described exemplary embodiments, it will be understood by those of ordinary skill in the art that modifications to, and variations of, the illustrated embodiments may be made without departing from the inventive concepts disclosed herein. Furthermore, disclosed aspects, or portions of these aspects, may be combined in ways not listed above. Accordingly, the invention should not be viewed as being limited to the disclosed embodiment(s).

What is claimed is:

1. An article of manufacture comprising:
a fiber-optic controller having:
a handle having a handle axis and a handle hollow that is defined throughout the handle along the handle axis, the handle comprising a collet with petals that extend along the handle axis and that have respectively-corresponding free distal ends and proximal ends,
wherein inner surfaces of the petals are separated from the first axis at locations of the free distal ends by a first distance and inner surfaces of the petals are separated from the first axis at locations of the proximal ends by a second distance smaller than the first distance; and
a nose cap having a nose cap axis and a nose cap hollow defined throughout the nose cap and extending along the nose cap axis,
wherein a portion of the nose cap hollow is a cavity extending along the nose cap axis and dimensioned such that when the handle and the nose cap are mated by inserting the free distal ends into the cavity and positioning a ridge of a first surface into a corresponding notch on a second surface, the free distal ends are brought towards one another to change the first distance to become substantially equal to the second distance,
wherein the first surface is one of an inner surface of the cavity and an outer surface of the handle while the second surface is the other of the inner surface of the cavity and the other surface of the handle.

2. An article of manufacture according to claim 1, wherein the cavity is dimensioned such that, when the handle and the nose cap are mated, a distance between an inner surface of a petal of the collet and the first axis is substantially constant at every point of said inner surface along the first axis for a given angular orientation in a plane perpendicular to the first axis.

3. An article of manufacture according to claim 1,
wherein each of the petals includes longitudinal ridges on a corresponding inner surface thereof, said longitudinal ridges having lengths that are substantially equal to a length of such petal,
wherein said longitudinal ridges are dimensioned such that, when a given optical fiber is pulled throughout the handle hollow and throughout the nose cap hollow and when the handle and the nose cap are mated, the longitudinal ridges are in contact with a plastic coating of said optical fiber substantially uniformly along the lengths of the longitudinal ridges while, at the same time, not being in contact with a cladding of the optical fiber.

4. An article of manufacture according to claim 3,
wherein an inner surface of the nose cap hollow is dimensioned to maintain a first non-zero separation from the plastic coating of the optical fiber when the handle and the nose cap are mated;
and/or
wherein an inner surface of the handle hollow is dimensioned to maintain a second non-zero separation from the plastic coating of the optical fiber when the handle and the nose cap are mated;
and/or
wherein the nose cap hollow includes a substantially circularly cylindrical portion and a transitional portion fluidly connecting the substantially circularly cylindrical portion with the cavity;
and/or
wherein the nose cap hollow includes a portion having a diameter that is substantially monotonically varying along a length thereof.

5. An article of manufacture according to claim 1,
wherein, said ridge is an arcual ridge as seen in a first plane transverse to a corresponding axis of the first and second axes, and said notch is a groove as seen in a second plane transverse to the other of the first and second axes.

6. An article of manufacture according to claim 1, devoid of a thread.

7. An article of manufacture according to claim 1,
having a handle surface indicia on an outer surface of the handle, said handle surface indicia configured to cause the outer surface of the handle to be asymmetric with respect to rotation about the handle axis; and/or
having a nose cap surface indicia on an outer surface of the nose cap, said nose cap surface indicia configured to cause the outer surface of the nose cap to be asymmetric with respect to rotation about the nose cap axis.

8. An article of manufacture according to claim 1, wherein a dimension of the nose cap hollow is not constant along a length of the nose cap and/or a dimension of the handle hollow is not constant along a length of the handle.

9. An article of manufacture according to claim 8, wherein at least one of the nose cap hollow and the handle hollow includes a substantially conical surface.

10. An article of manufacture according to claim 1, further comprising:
an optical fiber having a fiber core, a fiber cladding, and a plastic coating over the fiber cladding, said optical fiber positioned to be extended throughout the handle hollow and throughout the nose cap hollow.

11. An article of manufacture according to claim 10, further comprising an optical fiber termination device permanently cooperated with a distal end of the optical fiber such that the optical fiber termination device is separated from the fiber-optic controller by a non-zero length of said optical fiber.

12. An article of manufacture according to claim 11, wherein the optical fiber termination is configured to define an output axis along which light from the distal end of the optical fiber is outcoupled substantially transversely to the optical fiber.

13. An article of manufacture according to claim 12, wherein a spatial orientation of said output axis uniquely corresponds to a spatial orientation of a radius of the fiber-optic controller, said radius defined in a plane transverse to an axis of the fiber-optic controller when the handle and the nose cap are mated, said radius
(13A) connecting the axis of the fiber optic controller and the handle surface indicia; and/or
(13B) connecting the axis of the fiber-optic controller and the nose cap surface indicia.

14. A method comprising:
fabricating a fiber-optic controller that includes:
(i) a handle having a handle axis and a handle hollow that is defined throughout the handle along the handle axis, the handle comprising a collet with petals that extend along the handle axis and that have respectively-corresponding free distal ends and proximal ends,
wherein inner surfaces of the petals are separated from the first axis at locations of the free distal ends by a first distance and inner surfaces of the petals are separated from the first axis at locations of the proximal ends by a second distance smaller than the first distance; and
(ii) a nose cap having a nose cap axis and a nose cap hollow defined throughout the nose cap and extending along the nose cap axis,
wherein a portion of the nose cap hollow is a cavity extending along the nose cap axis and dimensioned such that when the handle and the nose cap are mated by inserting the free distal ends into the cavity and positioning a ridge of a first surface into a notch on a second surface, the free distal ends are brought towards one another to change the first distance to become substantially equal to the second distance,
by additive manufacturing or injection molding.

15. A method according to claim 14, further comprising:
cooperating
said handle, which has an optical fiber throughout the collet, with
said nose cap containing said optical fiber throughout the nose cap hollow
to position petals of the collet inside said nose cap hollow, while the optical fiber passes throughout both the handle and the nose cap; and
securing the nose cap onto the handle to form an optical fiber controller that contains the optical fiber by mutually repositioning the handle and the nose cap towards one another to insert the ridge into the notch,
thereby bringing a plastic coating of the optical fiber in contact with an internal surface of the collet substantially uniformly along a length of the petals while, at the same time, not bringing the petals in contact with a cladding of the optical fiber,
wherein the first surface is one of an inner surface of the hollow of the nose cap and an outer surface of the handle while the second surface is the other of the inner surface of the hollow of the nose cap and the other surface of the handle.

16. A method according to claim 15,
wherein said cooperating includes inserting the petals of the collet substantially completely into a first portion of the hollow of the nose cap while leaving a second portion of the hollow of the nose cap to contain only the optical fiber;
and/or
wherein the securing does not require a rotational movement of the nose cap and/or of the handle about the optical fiber;
and/or
wherein the securing is necessarily devoid of said rotational movement.

17. A method according to claim 15,
wherein at least one of an outer surface of the handle and an outer surface of the nose cap contains surface indicia causing said at least one of an outer surface of the handle and an outer surface of the nose cap to be rotationally asymmetric, and/or
wherein, when the optical fiber is a polarization maintaining fiber, the method further comprises pulling the optical fiber through the collet to have a fast axis or a slow axis of the optical fiber be angularly separated from said indicia in a plane transverse to an axis of the optical fiber, as seen along said axis, by a predetermined angle and/or to have said fast axis or a slow axis be substantially aligned with said indicia in said plane.

18. A method according to claim 15, wherein said ridge is an arcual ridge as seen in a first plane transverse to the optical fiber and/or said notch is a groove as seen in a second plane transverse to the optical fiber.

19. A method according to claim 15, wherein said bringing includes squeezing the plastic coating with longitudinal ridges of the inner surface of the collet without a longitudinal ridge of said longitudinal ridges touching the cladding to displace the plastic coating to provide resistance to rotation of the optical fiber inside the collet and/or to rotation of the collet about the optical fiber substantially at every point of said length.

20. A method according to claim 19, wherein the longitudinal ridges are substantially angularly equally distributed on the inner surface of the collet as viewed from an axis of the handle, wherein the longitudinal ridges are dimensioned to have the plastic coating cold flow in between the longitudinal ridges during said securing.

21. A method according to claim 15,
wherein each petal of the collet has a corresponding distal end and a corresponding proximal end, and
wherein the securing includes radially repositioning distal ends of the petals while keeping proximal ends of the petals substantially unmoved, thereby preventing a rotational movement of the optical fiber within the optical fiber controller substantially at every point of an inner surface of each petal.

22. A method according to claim 21, wherein:
(22A) the proximal ends are monolithically affixed to a body of the handle; and/or
(22B) a length of each of slits, separating the petals is substantially equal to the length of the collet; and/or
(22C) the longitudinal ridges are present at and extend along substantially all of each of the lengths of the petals.

23. A method according to claim 15, wherein at least one of the following conditions is satisfied:
(23A) the method further comprises affixing a fiber termination article to a distal end of the optical fiber, one of the nose cap and the handle is disposed between the distal end of the optical fiber and the other of the nose cap and the handle,
and/or
(23B) wherein said securing includes bringing the optical fiber in contact with the inner surface of the collet while keeping a gap between the optical fiber and an inner surface of the longitudinal hollow of the nose cap and/or an inner surface of the longitudinal hollow of the handle,
and/or
(23C) the method further comprises passing a portion of the optical fiber throughout the hollow of the handle and throughout the hollow of the nose cap, said portion covered with the plastic coating.

24. A method according to claim 23,
wherein, when the method further comprises affixing a fiber termination article to a distal end of the optical fiber:
(24A) said fiber termination article is configured to define an axis along which light from the distal end of the optical fiber is outcoupled substantially transversely to the optical fiber,
or
(24B) fiber termination article is configured to define an axis along which light from the distal end of the optical fiber is outcoupled substantially transversely to the optical fiber;
and
further comprising angularly rotating the nose cap and/or the handle about the optical fiber to redirect said axis.

25. A method according to claim 14, wherein said optical fiber controller does not contain any other component but the handle and the nose cap.
